(19) United States
(12) Patent Application Publication
(10) Pub. No.: US 2005/0119758 A1
(43) Pub. Date: Jun. 2, 2005

(54) Surgical implant for promotion of osseo-integration

(75) Inventors: Harold Alexander, Springfield, NJ (US); Bruce Hollander, Boca Raton, FL (US); John Ricci, Middleton, NJ (US)

Correspondence Address: MELVIN K. SILVERMAN, 500 WEST CYPRESS CREEK ROAD, SUITE 500, FT. LAUDERDALE, FL, 33309, US

(73) Assignee: BIO-LOK INTERNATIONAL INC.

(21) Appl. No.: 10/903,812

(22) Filed: Jul. 29, 2004

Related U.S. Application Data
(60) Provisional application No. 60/491,152, filed on Jul. 30, 2003.

(51) Int. Cl. A61F 2/28

(52) U.S. Cl. 623/23.5; 433/201.1; 623/23.76

(57) ABSTRACT

An implant for surgical insertion into tissue of a patient includes a microgeometric, repetitive pattern, in the form of a multiplicity of alternating ridges and grooves, each having an established width in a range of about 2 to about 25 microns, and an established depth in a range of about 2 to about 25 microns, each groove having a base and a wall; and a microgeometric random surface pattern, applied over the repetitive surface pattern, defining a multiplicity of micro-pits having dimensions in a range of about 0.1 to about 4 microns.

### BACKGROUND OF THE INVENTION

The present application is an improvement of our inventions set forth in U.S. Pat. Nos. 6,419,491 and 6,454,569 which relate to dental implants having surface textures that are adapted for the promotion of osseo-integration of an implant into surrounding bone.

As such, the present invention is also an improvement over prior art, such as U.S. Pat. No. 5,558,838 (1996) to Hansson, entitled Fixture For Use In a Dental System; U.S. Pat. No. 5,989,027 (1999) to Wagner, entitled Dental Implant Having Multiple Textured Surfaces; U.S. Pat. No. 4,553,272 (1985) to Mears, entitled Regeneration Of Living Tissues By Growth of Isolated Cells In Porous Implants; U.S. Pat. No. 5,607,607 (1997) to Naiman, entitled System and Assemblage for Producing Microtexturized Substrates and Implants; U.S. Pat. No. 5,833,641 (1998) to Curtis, entitled Wound Healing Material; and U.S. Pat. No. 5,976,826 (1999) to Singhvi, entitled Device Containing Cytophilic Islands; U.S. Pat. No. 4,320,891 (1982) to Branemark; and U.S. Pat. No. 5,571,017 (1996) to Niznick.

In the prior art, the focus has been on the use of random micro-pits, pores, or pods to enhance osseo-integration or, as in our above set forth prior inventions, the use of an ordered mircogeometric repetitive surface pattern in the form of alternating ridges and grooves. Although our said prior patents (see, for example, FIG. 7 of U.S. Pat. No. 6,419,491) suggest the possibility of the use of irregular horizontal surfaces with an ordered microgeometric repetitive surface pattern, the present invention further specifies the manner in which this may be accomplished to, thereby, address both random and non-random processes associated with interfaces and contact between surgical implants and surrounding hard and soft tissue of various types within a framework of the ordered microgeometric repetitive surface pattern.

### SUMMARY OF THE INVENTION

A surgical, typically metallic, implant may take the form of a solid elongate body including a longitudinal axis having distal and having proximal ends. Different portions thereof may include one or more different surface textures adapted for the promotion of tissue integration into the implant. In the case of a transcutaneous implant, such as a dental implant, certain sub-segments of the solid body may be provided with one subset to accommodate the integration of bone while another sub-segment is adapted for integration with surrounding soft tissue. However, in using one or more such sub-segments, all are provided with an ordered microgeometric repetitive pattern in the form of alternating ridges and grooves, each having an established x, y, and z-axis dimensions width in a range of about 2.0 to about 25 microns. Superimposed over said ordered repetitive surface pattern is a multiplicity of micro-pits having crater like characteristics to thereby provide roughness within and around the microgrooves. Such micro-pits exhibit surface and depth dimensions in a range of 0.1 to about 4 microns, not exceed the width of the microgrooves. The size of such micro-pits are however not sufficient to disrupt or disturb the dominant pattern of alternating ridges and grooves of the surface of the implant. Such micro-pits provide an attachment surface to “pods” or suction-cup like elements of cells of the tissue to be integrated.

It is accordingly an object of the invention to provide an improved microgeometric surface for surgical implants to alter and improve the osseo-integration of colonies of cells attached thereto.

It is another object to provide a combination of ordered and non-ordered microgeometric surfaces which are preferential to the growth of particular cell or tissue types.

It is a further object of the invention to provide a substrate for a microgeometric implant for the enhancement of in vivo cell attachment, orientation of cell growth and migration, and tissue function, such substrate having dimensions and geometry to prevent cell growth along a first or y-axis and for the inducement of cell growth along a second or x-axis.

It is a yet further object to provide a combination of repetitive and random microgeometric surface textures applicable to implants and a variety of other surgical applications.

The above and yet other objects and advantages of the present invention may become apparent from the hereinafter set forth Brief Description of the Drawings, Detailed Description of the Invention, and Claims appended herewith.

### BRIEF DESCRIPTION OF THE DRAWINGS

FIG. 1 is a plan diagrammatic view in an xy plane and at about 750 magnifications, showing ordered microgemetric surface patterns having parallel ridges and grooves, each of approximately equal width, in accordance with the present invention.

FIG. 2 is a view, similar to that of FIG. 1, however in which successive y-axis width of said ridges and grooves vary with y-axis direction of the surface pattern thereof.

FIG. 3 is a diagrammatic plan view of an ordered microgeometric surface pattern which defines a bi-axial, x-y matrix formed of alternating recesses and projections along each axis.

FIG. 4 is a plan view, similar to that of FIG. 3, however showing a pattern in which all recesses and projections thereof are co-linear with each other.

FIG. 5 is a plan view, similar to that of FIG. 4, in which all ridges are circular in x-y cross-section.

FIG. 6 is a view, similar to that of FIGS. 3 thru 5, in which the grooves of the pattern define an xy grid as the surface pattern thereof.

FIGS. 7 thru 14 are yz plane cross-sectional views of the patterns of FIGS. 1 thru 6 showing variations in yz plane geometry, that is, relationship of grooves to ridges that are applicable to one or more of the xy plane patterns shown in FIGS. 1 thru 6. A multiplicity of micro-pits randomly distribute on the grooves, the ridges and the walls.

FIGS. 15 thru 19 show further xy plane surface patterns which, respectively, comprise radiating, concentric, circular, radiating fan, radiating with concentric, and radiating with intersecting polar, patterns.

FIG. 20 is an in situ schematic view, at about 600 magnifications, showing a collar and proximal portion of a dental implant and tissue ingrowth associated therewith.

FIGS. 21 and 22 are enlarged views of another type of implant with which the present inventive microgeometric surface pattern may be employed.

FIG. 23 is an electron micrograph of a buttress thread type dental implant of the type of FIG. 20 showing the microgeometric structure at about 3000 magnifications.

FIG. 24 is an enlargement at about 340 magnifications of the collar portion of the implant of FIG. 20.

FIG. 25 is an electron micrograph, at about 3000 magnifications, showing use of discontinuous ridges and grooves, corresponding to the patterns shown in FIGS. 3, 6 and 19 above.

FIG. 26 is an electron micrograph, at about 3400 magnifications, of the views of FIGS. 27 and 28 below.

FIG. 27 is an electron micrograph, at about 3000 magnification of a surface pattern A or B upon the collar of the implant shown in FIGS. 20 and 24 in which the grooves thereof are continuous.

FIG. 28 is an electron micrograph, at 1200 magnifications, of the collar of the implant shown in FIGS. 20 and 24.

FIG. 29 is the xy plane, at about 750 magnifications, showing a further embodiment of the patterns of FIG. 1-2 above.

### DETAILED DESCRIPTION OF THE INVENTION

Bone tissue is the rigid supporting tissue constituting the principal component of almost all adult vertebrate skeletal structures. It exists in either dense or spongy form, known respectively as compact and cancellous bone. The typical bone cell size is of the order of about 10,000 nm, that is 10 microns.

Bone tissue consists of a chemical mixture of inorganic salts (65 to 70 percent) and various organic substances (30 to 35 percent) and is both hard and elastic. Its hardness is derived from inorganic constituents, principally calcium phosphate and calcium carbonate, with small amounts of fluorides, sulfates, and chlorides; its elasticity is derived from such organic substances as collagen, elastic cellular material, and fats. Internal tubular structures called Haversian canals contain nerve tissues and blood vessels that provide bones with organic nourishment. Surrounding these canals is a somewhat porous tissue composed of thin plates, known as lamellae, and usually containing cavities filled with a network of connective tissue called marrow or myeloid tissue. Bone marrow accounts for from 2 to 5 percent of the body weight of a person and consists of tissue of two types. Yellow bone marrow is made up principally of fat, and red bone marrow is tissue in which red and white blood cells and blood platelets originate. The external portions of bones, enclosing all the components mentioned above, include the compact and hardest of all bone tissue, which is in turn generally sheathed by a vascular, fibrous membrane known as the periosteum.

Micro-Texturing of Surface

With respect to bone and soft tissue adhering thereto, it has been found that the rate and direction of cell colony growth and the growth of different cell types surrounding surgical or dental implant can be controlled and effected by using the implants of this invention. In general, such implants comprise a plurality of separate zones of textured surface, each zone containing a different repetitive microgeometric design or pattern which is presented and exposed to the particular cell type for development of its unique colony growth. These different repetitive microgeometric textured design surfaces are intended to:
(a) promote the rate and orient the direction of bone growth, and discourage the growth of soft tissue to achieve secure fixation of the implant surface to bone tissue;
(b) promote the rate and orient the direction of the growth of soft tissue while discouraging the growth of bone tissue to achieve soft tissue integration with the implant surface; and/or
(c) create a barrier that discourages the growth of soft tissue, particularly soft fibrous tissue, and thereby prevent the migration of soft tissue growth in bone tissue attachment surfaces of the implant.

The implants of the invention can be provided from suitable and acceptable materials that are commercially available such as cast or wrought cobalt and chrome alloys, various grades of commercial titanium, titanium alloys, stainless steel alloys, thermoplastic resins such as polyethyletherketone, polyphenylene sulfide, ceramics, alumina, as well as combinations thereof.

A surface consisting of 12-μm groove and ridges has been shown to increase the RBM (rat bone marrow) to RTF (rat tendon fibroblast) cell colony growth ratio to encourage bone cell growth over fibrous tissue growth. In addition, this surface caused specific directional migration of bone cells at approximately twice the rate of cells on a flat surface. This surface can be used to enhance bone versus soft tissue growth as well as to direct bone growth into regions of an implant surface where bone fixation is needed.

Since fibrous tissue and bone cells generally “compete” for surface areas, the ratio of bone to soft tissue colony area increase, on a given surface, is an important parameter in surface selection. The ratio indicates the relative stimulation or inhibition of cell growth on these surfaces. Theoretically, this ratio would be significant to provide advantage for growth of one or another cell type on a surface, with high ratios favoring bone cell growth and low ratios favoring fibrous tissue growth. Based on these ratios, a 2-micron indentation or groove provided a 32.8% decrease in bone/soft tissue growth, providing a significant advantage in soft cell tissue growth. The surface could be used to increase fibrous tissue cell growth; it can also be used to significantly orient growth of these cells. A 4-micron indentation or groove surface provided a similar ratio, but it is based on lower overall growth rates. Therefore, if non-oriented fibrous cell growth is required, a flat control surface provides an inherent advantage to RTF tissue cells at a ratio of bone to soft tissue cell growth of approximately 0.6. This effect has been observed in vivo where smooth surfaces have been shown to favor formation of thick fibrous tissue capsule formation as compared to textured surfaces of the same composition, which show less fibrous capsule formation and more extensive osteointegration.

The surface having the highest ratio of bone to soft tissue cell growth is the 12-μm/micron indentation or groove substrate.

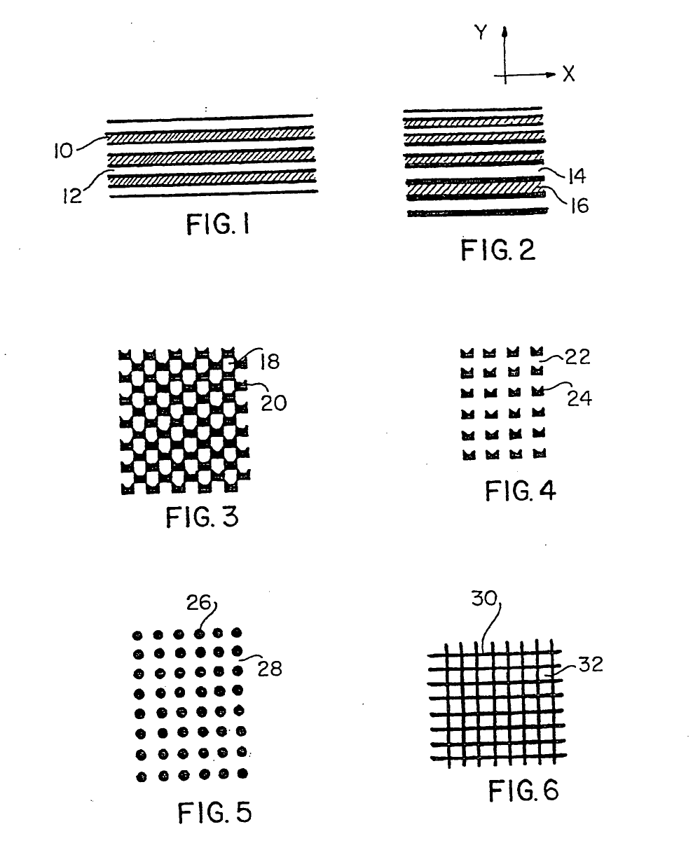

With reference to FIG. 1, the subject ordered microgeometric repetitive patterns may take the form of a multiplicity of alternating grooves 10 and ridges 12 in which each respective ridge and groove displays a width between about 6.0 to about 25 microns and a depth in a range between about 2 to about 25 microns. In the embodiment of FIG. 1, an infinite repeating pattern of co-parallel linear ridges and grooves having substantially equal width defines a micro textured surface of an implant or substrate as contemplated by the instant invention.

In the embodiment of FIG. 2 is shown a surface in which alternating ridges 14 and grooves 16 increase y-axis in width with reference to a transverse axis relative to the axis of said ridges and grooves. Accordingly, with reference to types of tissues with which a transition of tissue type or gradient of tissue density exists, a textured surface of the type of FIG. 2 may be employed.

In FIG. 3, is shown a surface pattern in which ridges 18 take the form of projections while grooves 20 take the form of recesses to thereby define a checkerboard configuration. Therein such ridges and grooves alternate with reference to both a x and y axes of a given surface.

The embodiment of FIG. 4 differs from that of FIG. 3 in that ridges 22 thereof form a bi-axial linear pattern. Similarly, grooves 24 of the embodiment of FIG. 4 define a x-y matrix formed of recesses that may assume a number of geometries.

In FIG. 5 is shown embodiment of the invention in which circular depressions 26 define grooves or depressions while the areas therebetween, namely, spaces 28 define ridges or projections. It may, therefrom be appreciated that the terminology “alternating ridges and grooves,” as used herein, encompasses a variety of microtexturized geometric patterns in which the ridges and grooves thereof while alternating relative to each other may themselves comprise any one of a variety of geometries inclusive of channels, rectangles, parallelograms, squares, circles and ovals.

With reference to FIG. 6, there is shown a grid like arrangement in which grooves 30 define an xy matrix which is etched into a surface 32 such that surface 32, when viewed relative to etched grooves 30, comprises ridges.

From the embodiment of FIGS. 1 thru 6 it may be appreciated that the width (or diameter) of a given groove need not correspond to that of its respective ridge, providing such widths fall within the above-referenced range of about 2 to 25 microns with a depth in a range of about 2 to about 25 microns. It has, thereby, through extensive experimentation as set forth above, been determined that a micro-geometric repetitive pattern within the scope of the present invention may define a guide for preferential promotion of the rate, orientation and directionality of growth of colonies of cells of maxillofacial bone or tissue without requirement that the width of a ridge be equal to that of a groove in that it is, essentially, the groove of the microtexturized surface that defines the guide for preferential promotion of growth of colonies of cells. In most applications, it is desirable to maximize the density of grooves upon a given surface to thereby attain the desired cell growth effect; however, differing clinical environments will dictate use of different surface patterns and density of distribution of grooves.

It is to be understood that, for clarity, FIGS. 1-6 do not show the below-described use of random micro-pits over said groove structure.

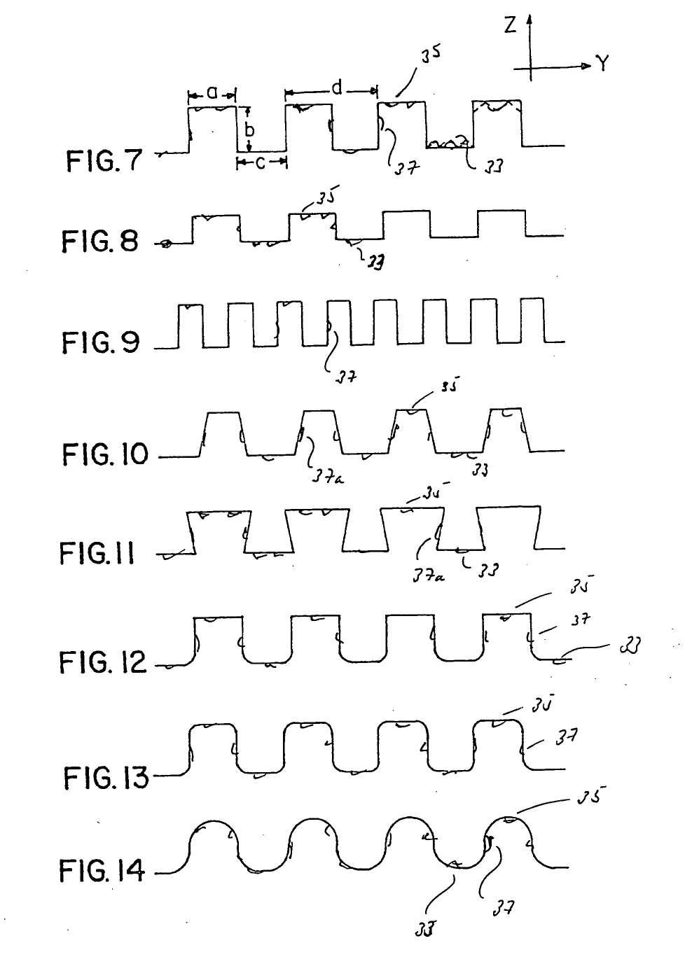

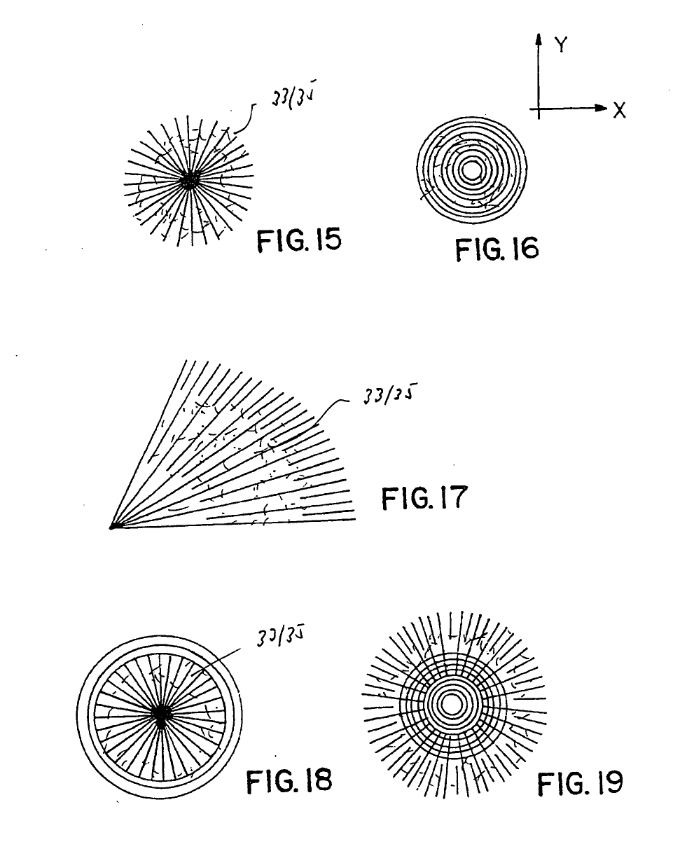

With reference to the views of FIGS. 7 thru 14, there is shown diagrammatic cross-sections which may be employed in association with the microgeometric textured configurations above described with reference to FIGS. 1 thru 6. In other words, the views of FIGS. 7 thru 14 illustrate the range of geometries which may be defined within the yz plane of the surface patterns. Resultingly, FIGS. 7 thru 9 show variations in ridge width a, ridge and groove height b, and groove width c. Typically, ridge height will equal groove depth. Parameter d is the sum of ridge and groove width. The ridge surface of the right-most of FIG. 7 indicates that y-axis surfaces need not be linear flat, that is, may be irregular, micro-pitted or crater-like.

In FIGS. 7 thru 14, micromechanical pits 33 and 35, each having a dimension in a range of 0.1 to about 4 microns, as is shown upon upper and lower y surfaces “a” and “c” of the microgrooves structures thereof. In addition, microgrooves 37 are shown upon the vertical (z-axis) surfaces “b” of the patterns of FIGS. 7-9, and 12-14. Similar micro-pits, craters or pores 37a may be placed upon the angled sidewalls of the geometries shown in FIGS. 10 and 11. Said micro-pits facilitate attachment of “pods” of the tissue cell wall to the implant surface.

In the geometries of FIGS. 15-19, xy plane micro-pits 33/35 are shown as dotted and dashed lines. It is accordingly to be appreciated that the micro-pits are typically provided in a substantially random fashion over the underlying xy plane of ordered microgrooves and ridges shown in FIGS. 1-6 and 15-19.

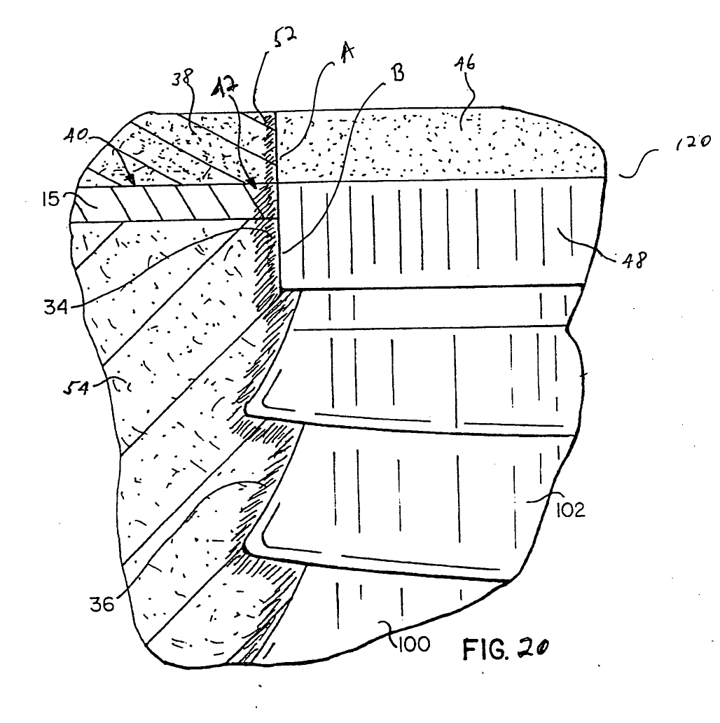

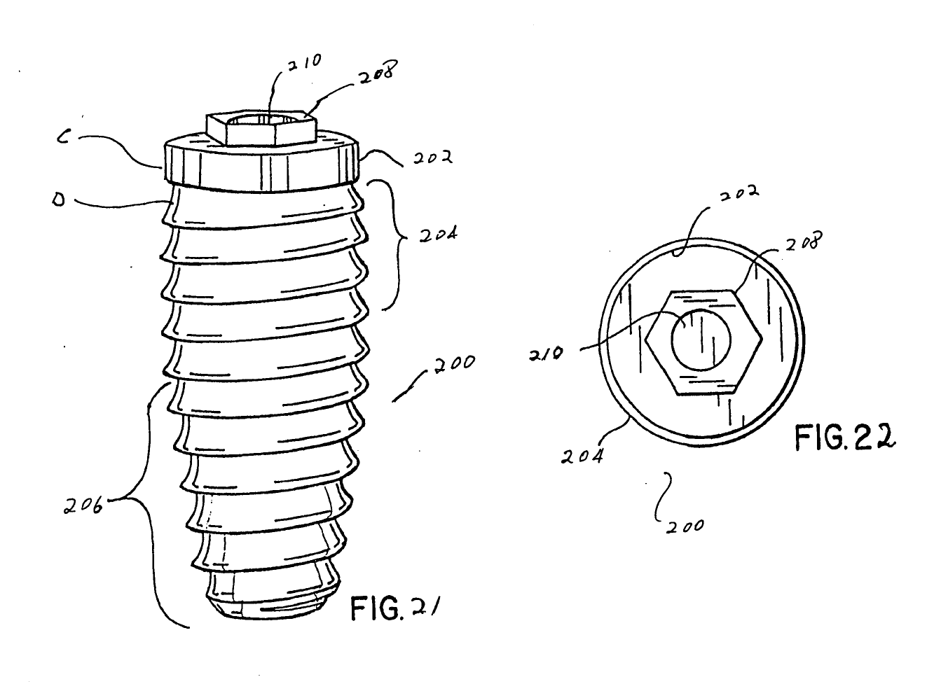

With reference to FIG. 20, there is shown an example in which the above surface treatments of medical implants may be applied in a dental application. More particularly, in FIG. 20 is shown an enlargement of a collar 120 having a proximal collar segment 46 and a distal collar segment 48 of a buttress thread implant 100, the same relative to jaw bone 54, cortical bone 15, and soft tissue 38. Also shown in FIG. 20 is a region 34 of osseo-integration between said distal collar segment 48 and a bone 54 as well as a region 36 of osseo-integration between distal region 102 of the implant 100 and bone 54. In region 42 is shown an area of integration between cortical bone 15 and distal collar segment 48. Area 52 represents a region of osseo-integration between proximal collar segment 46 and soft tissue (gum) 38. These regions of ingrowth are enabled by the use of a smaller dimension microgeometric pattern B for bone integration and a larger dimension pattern A for soft tissue sealing, this within the above referenced range of about 2.0 to about 25 microns as the width and depth of the alternating ridges 12/14 and grooves 10/16 (see FIGS. 1, 2, and 7-14), with random micro-pits which define the ordered microgeometric repetitive surface pattern of the inventive substrate.

It is therefore to be appreciated that regions 34, 36, 42 and 52 of ingrowth or bioaffinity between jawbone 54, cortical bone 15, and tissue 38, and collar segments 46 and 48, and distal region 102 accomplish an advantageous sealing of the tissue about area 42 of interface 40 between tissue 38 and cortical bone 15, i.e., at the point of entry of the implant collar into said bone. As such, a dual affinity implant collar, in accordance with the present invention, effectively promotes sealing of bone 42 to implant collar 120. With such sealing, the so-called cupping effect, a longstanding problem in the prior art of implant dentistry, is precluded.

It should be further appreciated that the above described-substrate pattern, comprising a combination of ordered microgeometric alternating ridges and grooves having dimensions in the range of about 2.0 to about 25 microns, with an overlay of substantially random micro-pits having a dimension in a range of about 0.1 to about 4 microns, may be affected by any one of a number of means including, without limitation, the following:

Laser cutting, acid etching, photolithography, abrasion/roughening, plasma spraying, calcium sulfate, biocompatible glass, collagen, hydroxapatite, growth factor compounds, and combinations thereof.

With respect to the ratio of axial length of the proximal to the distal segments of the collar, it has been found that such axial lengths need not necessarily be equal, such that a range of axial length of the proximal to the distal segments may fall between about 1:4 to about 4:1, this within an aggregate axial length of between about 1 to about 3 millimeters.

With reference to FIGS. 21-22, there is shown an implant 200 having an enlarged proximal segment 204, as is taught in our U.S. Pat. No. 6,406,296, to which the above set forth surface pattern may be applied. Such an implant also includes a collar 202, a tightening head 208, engagement means 210 therein, and a tapered distal portion 206 thereof. To promote tissue ingrowth and sealing as in the manner above described with reference to FIG. 20, one surface pattern C can be applied to collar 202 while another surface pattern D can be applied to said enlarged proximal segment 204. Thereby, both the enlarged proximal portion 204 and the microgeometric substrates C and D interact to enhance osseo-integration at the site of the implant.

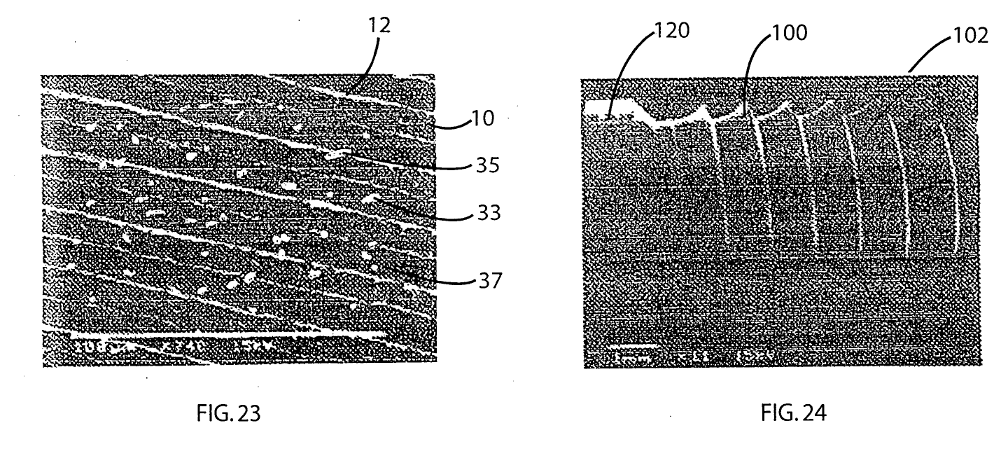

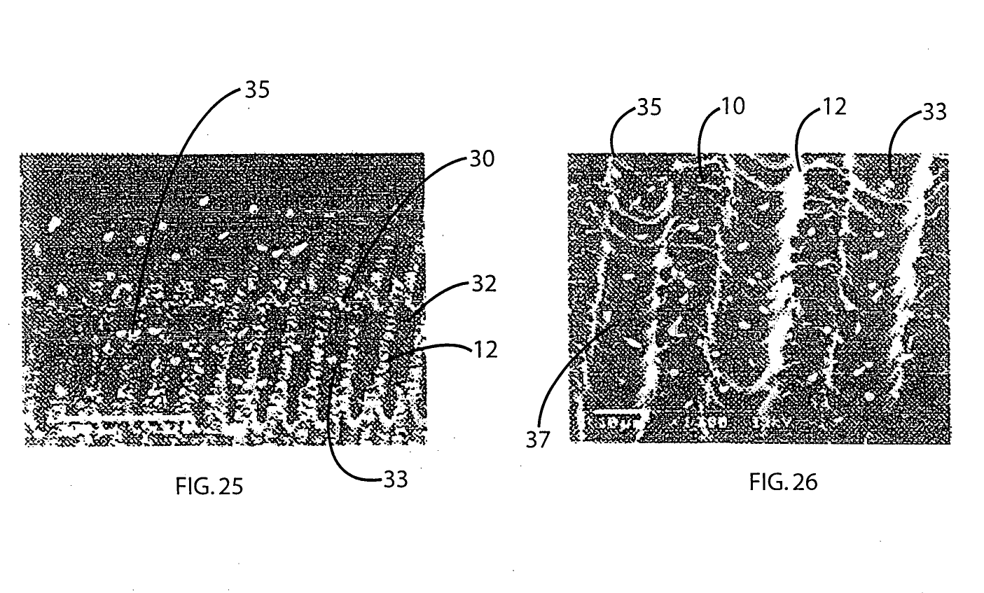

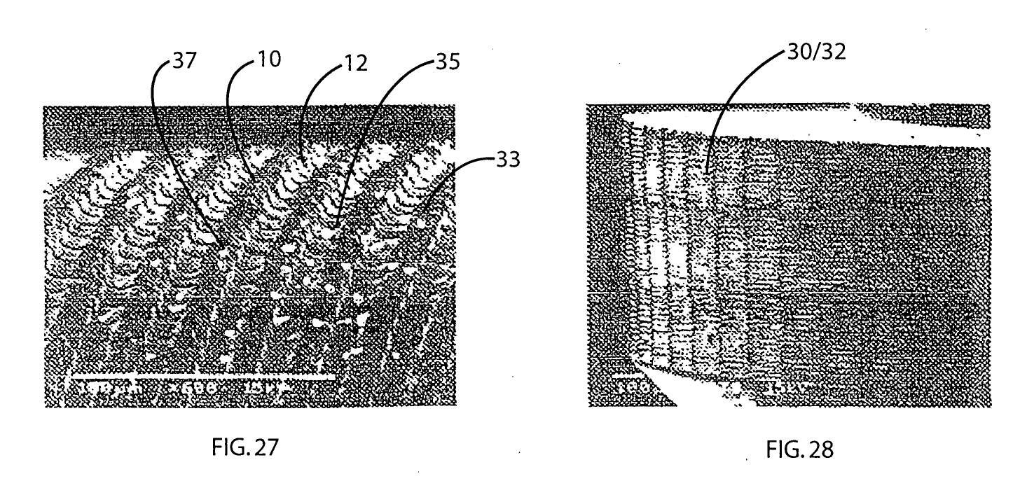

FIG. 23 is an enlarged view of a buttress thread dental implant, of the type of FIG. 24, which has been provided with the ordered microgeometric surface. FIG. 25 is an enlargement at 340 magnifications of the collar portion of FIG. 20, however showing a pattern of discontinuous grooves 30 and ridges 32, as depicted in FIG. 6 previously. FIG. 26 is an electron micrograph comprising a further enlargement of the collar of FIG. 25. FIG. 27 is an electron micrograph of the surface pattern upon the thread structure of the implant of FIG. 24 in which the grooves thereof are continuous, as opposed to the discontinuous ridge and groove segments of FIG. 25. FIG. 28 is a 1200-power electron micrograph enlargement of the collar of the implant shown in FIG. 24. In all figures, the small longitudinal grooves therein reflect laser-related melting, rather than a part of the microgeometric surface of the implant.

Also shown in all micrographs are micro-pits (pods) 33, 35 and 37, described above with reference to FIGS. 7-19.

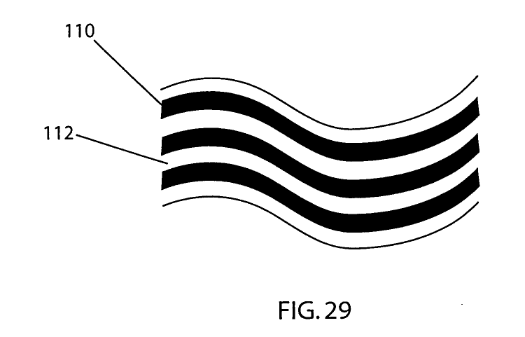

Shown in FIG. 29 is a further embodiment of the invention on which grooves 110 and ridges 112 define parallel but curvilinear lines.

While there has been shown and described the preferred embodiment of the instant invention it is to be appreciated that the invention may be embodied otherwise than is herein specifically shown and described and that, within said embodiment, certain changes may be made in the form and arrangement of the parts without departing from the underlying ideas or principles of this invention as set forth in the Claims appended herewith.

1. A medical implant system comprising an implant element for surgical insertion into tissue of a patient, said implant element comprising:
(a) a microgeometric, repetitive pattern, in the form of a multiplicity of alternating ridges and grooves, each having an established width in a range of about 2 to about 25 microns, and an established depth in a range of about 2 to about 25 microns, each groove having a base and two sidewalls; and
(b) a microgeometric random surface pattern, applied over said repetitive surface pattern, defining a multiplicity of micro-pits having dimensions in a range of about 0.1 to about 4 microns.

2. The system of claim 1, wherein said dimensions of said multiplicity of micro-pits do not exceed said width of said grooves, and said depth of said grooves.

3. The system of claim 2, wherein said multiplicity of micro-pits randomly distribute on said base and said sidewalls of said each groove.

4. The system of claim 2, wherein said multiplicity of micro-pits randomly distribute on an upper surface of said ridges.

5. The system of claim 2, wherein said each groove defines, in radial cross-section, a relationship of said base to one of said sidewalls equal to, or less than, about 90 degrees.

6. A medical implant for surgical insertion into an implant site of a patient, said medical implant comprising:
(a) an ordered microgeometric surface pattern in the form of a multiplicity of alternating ridges and grooves; each of said alternating ridges and grooves having a width in a range of about 2 to about 25 microns, and a depth in a range of about 2 to about 25 microns; and each of said grooves having a base and two sidewalls; and
(b) a microgeometric random surface pattern in the form of a multiplicity of micro-pits having dimensions in a range of about 0.1 to about 4 microns, superimposed over said ordered microgeometric surface pattern.

7. The medical implant of claim 6, wherein said dimensions of said multiplicity of micro-pits do not exceed said width of said grooves, and said depth of said grooves.

8. The medical implant of claim 7, wherein said multiplicity of micro-pits randomly distribute on said base and said sidewalls of said grooves.

9. The medical implant of claim 7, wherein said multiplicity of micro-pits randomly distribute on an upper surface of said ridges.

10. The medical implant of claim 7, wherein said multiplicity of alternating ridges and grooves have a substantially same width in a range of from about 2 to about 25 microns.
